(19) United States
(12) Patent Application Publication
(10) Pub. No.: US 2023/0310301 A1
(43) Pub. Date: Oct. 5, 2023

(54) METHODS AND PROCESSES FOR TREATING THE GLABELLA

(71) Applicants: SEBASTIAN COTOFANA, Rochester, MN (US); CLAUDIA ANDREA HERNANDEZ, Medellin (CO)

(72) Inventors: SEBASTIAN COTOFANA, Rochester, MN (US); CLAUDIA ANDREA HERNANDEZ, Medellin (CO)

(21) Appl. No.: 17/712,951

(22) Filed: Apr. 4, 2022

(51) Int. Cl. A61K 8/66; A61Q 19/08

(52) U.S. Cl. CPC A61K 8/66; A61K 2800/91; A61Q 19/08

(57) ABSTRACT

Methods for improving the aesthetic appearance of a human face in the glabella by targeting horizontal and vertical glabellar lines are provided. The methods use a 3-point injection algorithm which is based exclusively on the anatomy of the procerus and corrugator supercilii muscles. The methods and processes of injection taught herein target the bony origin of the procerus and corrugator supercilii muscles without additional product administration above the level of the hairy eyebrow.

### FIELD OF THE INVENTION

This patent document relates generally to methods and processes for enhancing the aesthetic appearance of a human face. More particularly, the subject matter of this patent document relates to enhancing the aesthetic appearance of a human face at the glabella through neuromodulator treatments.

### BACKGROUND

Neuromodulator treatments of the face account for 48.2% of all non-surgical aesthetic procedures worldwide with more than 6 million injections performed in 2018, according to the global survey of the International Society of Aesthetic Plastic Surgery (ISAPS). Toxin injection techniques vary depending on the facial region treated and manufacturer guidelines, which offer predefined injection points to guide experts and novices toward predictable outcomes.

Current injection algorithms for treating the glabella rely on a five (5) or seven (7) point injection techniques, which target the procerus and the medial and the lateral aspects of the corrugator supercilii muscles; the latter is injected above the eyebrow. The reported incidence rates for eyebrow ptosis were reported to be at 3.1% and to be independent of the product administered. The explanatory model for this adverse event is that the eyebrow elevation segment of the frontalis muscle is affected, which results in an inability to elevate the eyebrow and to act as an antagonist for the eyebrow depressors at rest. Although eyebrow ptosis is not long-lasting, it can cause significant patient dissatisfaction and should be avoided. However, current injection algorithms target the lower forehead despite its close proximity to the upper margin of the eyebrow. A deeper understanding of the underlying anatomy may suggest an injection algorithm which would not administer neuromodulators cranial to the level of the hairy eyebrows.

Accordingly, one object of the methods and process taught herein is to provide a treatment technique for the horizontal and vertical lines in the glabella that is more effective. Another object of the methods and process taught herein is to provide a treatment technique that reduces undesirable side-effects, for example, eyebrow ptosis.

### SUMMARY

Methods and processes for treating horizontal and vertical lines in the glabella are provided. In preferred embodiments, the method of treating horizontal and vertical lines in a glabella comprises a first injection of a first dosage of neuromodulator in the glabella at a midline and at a level of a connecting line between the left and right medial canthal ligaments. For the first injection, an injecting device is inserted to a depth such that the injecting device is in contact with the nasal bone and the first injection is done in constant direct contact with the nasal bone.

A second injection of a second dosage of neuromodulator in the glabella is then performed. The second injection is located at the corrugator muscle at the medial and inferior margin of the left eyebrow. The injecting device is inserted to a depth such that the injecting device is in contact with the underlying frontal bone and the second injection is done in constant contact with the frontal bone.

Finally, a third injection of a third dosage of neuromodulator in the glabella if performed. The third injection is located at the corrugator muscle at the medial and inferior margin of the right eyebrow. The injecting device is inserted to a depth such that the injecting device is in contact with the underlying frontal bone and the third injection is done in constant contact with the frontal bone.

In preferred embodiments, the first injection is done perpendicular to the surface of the nasal bone or close thereto. In addition, the second injection and third injection are both done at a 45-degree angle to the midline or close thereto.

In preferred embodiments of the techniques taught herein, only the three injections are needed and performed.

Preferably, the administering doctor selects the appropriate dosage for each injection based on the needs of the patient, as is known in art. In some embodiments, the dose for the first injection is 5.23±2.5 International Units and the dose for the second and third injections is 13.27±5.7 International Units.

Any neuromodulator may be used but in preferred embodiments, the neuromodulator is selected from the group consisting of onabotulinum toxin type A, incobotulinum toxin type A, Prabotulinumtoxin toxin type A, and abobotulinum toxin type A.

### BRIEF DESCRIPTION OF THE DRAWINGS

FIG. 1 is a face on view of a human head showing three injection sites for use in the methods and process for treating the glabella described herein.

### DETAILED DESCRIPTION

Recent facial anatomic research findings provide for a more accurate estimation of the underlying muscular anatomy from skin surface landmarks, thus providing additional information on which to base neuromodulator treatment algorithms. In 2017, a cadaveric study related the shape of the horizontal forehead lines to the underlying muscle fascicle angle of the frontalis muscle and formed a fundament for skin surface relationships. These results were confirmed in 2019 in a clinical study utilizing skin vector displacement technology and revealed that the shape of horizontal forehead lines are statistically significantly related to the direction of forehead skin movement. Utilizing the same technology, in 2020 the bi-directional movement of the frontalis muscle was described and an explanatory model for eyebrow ptosis after neuromodulator application in the lower part of the forehead was proposed. The inventors introduced the “Line of Convergence” which separated the frontalis muscle into an eyebrow elevation segment (=lower forehead) and into a hairline-depression segment (=upper forehead). This segmentation of the frontalis muscle provides a mechanism by why neuromodulators injections into the lower forehead may result in eyebrow ptosis.

The present patent document describes unique and inventive methods for improving the aesthetic appearance of a human face in the glabella by targeting horizontal and vertical glabellar lines. The methods use a 3-point injection algorithm which is based exclusively on the anatomy of the procerus and corrugator supercilii muscles. The methods and process of injection taught herein target the bony origin of the procerus and corrugator supercilii muscles without additional product administration above the level of the hairy eyebrow.

One strength of the methods and processing taught herein is the innovative approach to determine the 2- and 3-dimensional location of the injection points based purely on anatomy and on the muscular origin of the targeted muscles. Despite the plethora of currently available injection points and algorithms, the presented injection technique is unique at least because it relies exclusively on targeting the origins of the muscles responsible for glabellar frown lines.

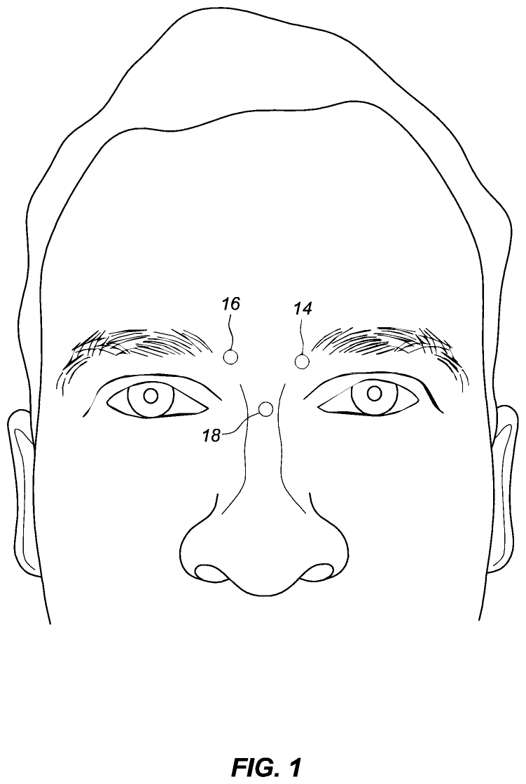

FIG. 1 illustrates a front plan view of a face showing the three injection points 14, 16 and 18 of the methods taught herein. The position of the three injection points matches precisely with the anatomic origin of the procerus and the corrugator supercilii muscles.

In the processes taught herein, the three injection sites 14, 16 and 18 receive a single injection each. As used herein, “single injection” means per treatment such that if a patient comes back some significant time later and needs another treatment an additional injection at teach site may be performed.

Each site received a single injection of a neuromodulator. Examples of neuromodulators that may be used herewith include but are not limited to: onabotulinum toxin type A, Allergan, incobotulinum toxin type A, Merz Pharma; abobotulinum toxin type A and Prabotulinum toxin type A. In other embodiments other neuromodulators may be used.

The first injection site 18 is in the midline at the level of a connecting line between the left and right medial canthal ligaments. In preferred embodiments, the first injection is done at a perpendicular angle to the nasal bone surface. In preferred embodiments, the injecting device is inserted to a depth such that it is in contact with the nasal bone and the first injection is done in constant direct contact with the nasal bone.

The second and third injection sites 14 and 16 target the corrugator muscles at the medial and inferior margin of the eyebrows. A single injection of neuromodulator is done at each site 14 and 16. In preferred embodiments, the injection at each site 14 and 16 is done at a 45-degree angle in relation to the midline. In other embodiments, other angles of injection may be used, for example, between 30 degrees and 60 degrees in relation to the midline. However, 45-degrees to the midline is preferred.

In preferred embodiments, each injection is done to the depth of the underlying frontal bone and bone contact is maintained during the injection procedure. No additional injections other than the three described are used.

As one skilled in the art will appreciate, the ideal angles are perpendicular and 45-degrees but in practice, the injections may differ from those angles and still be within the scope of the embodiments taught herein. Ideally, the injections are about perpendicular and about 45-degrees such that the injection are at those angles±10 degrees.

In preferred embodiments, the treating physician can choose the amount of neuromodulator units injected at each location based on the patient's anatomy and needs. Factors typically considered are a patient's variation in muscular anatomy, soft tissue thickness, age, gender and ethnic background, to name a few. In a study of 105 patients, the first injection site 18 for treating the procerus muscle received a mean dose of 5.23±2.5 International Units (I.U.) (12.90±6.30 Speywood Units (S.U.). In the same study, the second and third doses at the injection sites 14 and 16 for treating each corrugator supercilia muscle were 13.27±5.7 I.U. (33.17±14.2 S.U.). In the study, the dosage (measured in international units and in Speywood units) injected into the procerus and into the left/right corrugator supercilii muscles were converted with 1:1 for onabotulinum toxin type A, 1:1 for incobotulinum toxin type A and 1:2.5 for abobotulinum toxin type A.

No injections are performed at the upper margin or above the hairy eyebrow. The injections are performed only inside or inferior to the eyebrow. When using the methods taught herein, reduction of the horizontal and vertical lines of the glabella may be achieved with no eyebrow ptosis and no upper eyelid ptosis.

The bony origin of the procerus muscle is located in the midline of the nasal bone whereas its insertion is the dermis and the frontalis muscle at the level of the upper margin of the medial heads of the eyebrows. The muscle majorly determines the position of the medial eyebrows and is at a muscular balance with the eyebrow elevating frontalis muscle. Injections cranial to the superior margin of the eyebrows can therefore affect the eyebrow elevation segment of the frontalis muscle and result in medial eyebrow ptosis. This is a relatively frequent adverse event after forehead and glabella neuromodulator treatments and can also be seen with a hyper-elevation of the lateral eyebrow. Targeting the origin of the procerus muscle exclusively at its bony insertion eliminates the possibility of medial eyebrow ptosis as no frontalis muscles will be affected.

The corrugator supercilii muscle originates from the superciliary arch of the frontal bone around 0.5 cm lateral to the midline slightly medial and inferior to the most common location of the supratrochlear notch/foramen. From this location the muscle courses laterally and cranially to insert in the middle and/or lateral third of the skin of the eyebrow. Its cranial boundary is formed by the inferior frontal septum which corresponds to the upper margin of the hairy eyebrow. At its dermal insertion, orbicularis oculi and frontalis muscle fibers merge and connect additionally to the overlying skin comparable to the peri-oral subcutaneous architecture. In the past, most commonly performed injection techniques target this muscle in two or three locations of which the medial injection is aimed at the muscle belly and the lateral injection at its dermal insertion above the eyebrow or even further lateral and cranial. The lateral injections have the potential to drop the eyebrow as the eye brow elevating segment of the frontalis muscle can be affected resulting in an unbalance favoring the inferior-pull of the eyebrow depressors.

In the methods and processes taught herein the injections at sites 14 and 16 targeted exclusively the bony origin of the corrugator supercilii muscle without a lateral injection component. This results in an elevation of the lateral head of the eyebrow in the resting position. This can be explained by the reduction of the medial and inferior-pull of the corrugator supercilii muscle which favored the increase in eyebrow height after the onset of the neuromodulator effect. The eyebrow-elevation segment of the frontalis muscle is not affected by the treatment (medial injection only).

The 3-point injection techniques taught herein specifically target the bony origin of the procerus and corrugator supercilii muscles resulting in predictable and long-lasting effects up to 4-months. Due to the specific administration of toxin exclusively at the bony origin, collateral (adverse) effects are reduced or eliminated. Eyebrow and upper eyelid ptosis is eliminated or reduced as the eyebrow-elevation segment of the frontalis muscle is not affected. No injections cranial to the upper margin of the hairy eyebrow are needed. The central portion of the frontalis muscle is not affected which can result in a hyper-active contraction state of the lateral frontalis muscle fibers presenting clinically as a “Spock” or “Mephisto” eyebrow shape. This was avoided by the injection at the bony origin of the procerus muscle which leaves the medial frontalis muscle activity unaffected.

Although the various inventive aspects are herein disclosed in the context of certain preferred embodiments, implementations, and examples, it will be understood by those skilled in the art that the present invention extends beyond the specifically disclosed embodiments to other alternative embodiments and/or uses of the invention and obvious modifications and equivalents thereof. In addition, while a number of variations of the inventive aspects have been shown and described in detail, other modifications, which are within their scope will be readily apparent to those of skill in the art based upon this disclosure. It should be also understood that the scope this disclosure includes the various combinations or sub-combinations of the specific features and aspects of the embodiments disclosed herein, such that the various features, modes of implementation, and aspects of the disclosed subject matter may be combined with or substituted for one another. Thus, it is intended that the scope of the present invention herein disclosed should not be limited by the particular disclosed embodiments or implementations described above, but should be determined only by a fair reading of the claims.

Similarly, this disclosure is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

Further, all claim terms should be interpreted in their most expansive forms so as to afford the applicant the broadest coverage legally permissible. Although the embodiments have been described with reference to the drawings and specific examples, it will readily be appreciated by those skilled in the art that many modifications and adaptations of the processes, methods and apparatuses described herein are possible without departure from the spirit and scope of the embodiments as claimed herein. Thus, it is to be clearly understood that this description is made only by way of example and not as a limitation on the scope of the embodiments as claimed below.

What is claimed is:

1. A method of treating horizontal and vertical lines in a glabella comprising:
a first injection of a first dosage of neuromodulator in the glabella at a midline at a level of a connecting line between the left and right medial canthal ligaments wherein a first injecting device is inserted to a first depth such that the first injecting device is in contact with a nasal bone and the first injection is done in constant direct contact with the nasal bone;
a second injection of a second dosage of neuromodulator in the glabella at a corrugator muscle at a medial and inferior margin of a left eyebrow wherein a second injecting device is inserted to a second depth such that the second injecting device is in contact with an underlying frontal bone and the second injection is done in constant contact with the frontal bone; and
a third injection of a third dosage of neuromodulator in the glabella at a corrugator muscle at a medial and inferior margin of a right eyebrow wherein a third injecting device is inserted to a third depth such that the third injecting device is in contact with an underlying frontal bone and the third injection is done in constant contact with the frontal bone.

2. The method of claim 1, wherein exactly three injections are performed.

3. The method of claim 1, wherein the first injection is done about perpendicular to a surface of the nasal bone.

4. The method of claim 3, wherein the second injection and third injection are done at about a 45 degree angle to the midline.

5. The method of claim 1, wherein the first dose is 5.23±2.5 International Units.

6. The method of claim 5, wherein the second dose is 13.27±5.7 International Units.

7. The method of claim 6, wherein the second dose is 13.27±5.7 International Units.

8. The method of claim 1, wherein the neuromodulator is selected from the group consisting of onabotulinum toxin type A, incobotulinum toxin type A, Prabotulinum toxin type A and abobotulinum toxin type A.

9. A method of treating horizontal and vertical lines in a glabella comprising:
a first injection of a first dosage of neuromodulator in the glabella at a midline at a level of a connecting line between a left and right medial canthal ligaments wherein a first injecting device is inserted to a first depth such that the first injecting device is in contact with a nasal bone and the first injection is done in constant direct contact with the nasal bone and about perpendicular to a surface of the nasal bone;
a second injection of a second dosage of neuromodulator in the glabella at a corrugator muscle at a medial and inferior margin of a left eyebrow wherein a second injecting device is inserted to a second depth such that the second injecting device is in contact with an underlying frontal bone and the second injection is done in constant contact with the frontal bone and at about a 45-degree angle to the midline; and
a third injection of a third dosage of neuromodulator in the glabella at a corrugator muscle at a medial and inferior margin of a right eyebrow wherein a third injecting device is inserted to a third depth such that the third injecting device is in contact with an underlying frontal bone and the third injection is done in constant contact with the frontal bone and at about a 45-degree angle to the midline.

10. The method of claim 9, wherein only the first injection, second injection and third injection are performed.

11. The method of claim 9, wherein the first dose is 5.23±2.5 International Units.

12. The method of claim 11, wherein the second dose is 13.27±5.7 International Units.

13. The method of claim 12, wherein the second dose is 13.27±5.7 International Units.

14. The method of claim 9, wherein the neuromodulator is selected from the group consisting of onabotulinum toxin type A, incobotulinum toxin type A, abobotulinum toxin type A and Prabotulinum toxin type A.

15. A method of treating horizontal and vertical lines in a glabella comprising:
a first injection of a first dosage of neuromodulator in the glabella at a midline at a level of a connecting line between a left and right medial canthal ligaments wherein a first injecting device is inserted to a first depth such that the first injecting device is in contact with a nasal bone and the first injection is done in constant direct contact with the nasal bone and about perpendicular to a surface of the nasal bone;
a second injection of a second dosage of neuromodulator in the glabella at a corrugator muscle at a medial and inferior margin of a left eyebrow wherein a second injecting device is inserted to a second depth such that the second injecting device is in contact with an underlying frontal bone and the second injection is done in constant contact with the frontal bone and at about a 45-degree angle to the midline; and
a third injection of a third dosage of neuromodulator in the glabella at a corrugator muscle at a medial and inferior margin of a right eyebrow wherein a third injecting device is inserted to a third depth such that the third injecting device is in contact with an underlying frontal bone and the third injection is done in constant contact with the frontal bone and at about a 45-degree angle to the midline; and
wherein the neuromodulator is selected from the group consisting of onabotulinum toxin type A, incobotulinum toxin type A, Prabotulinum toxin type A and abobotulinum toxin type A.

16. The method of claim 15, wherein exactly three injections are performed.

17. The method of claim 15, wherein the first dose is 5.23±2.5 International Units.

18. The method of claim 17, wherein the second dose is 13.27±5.7 International Units.

19. The method of claim 18, wherein the second dose is 13.27±5.7 International Units.
